(19) United States
(12) Patent Application Publication
(10) Pub. No.: US 2012/0209220 A1
(43) Pub. Date: Aug. 16, 2012

(54) Pocket douche and Enema

(76) Inventors: Russell Uy, Daly City, CA (US); Liberty Bernardino Uy, Daly City, CA (US)

(21) Appl. No.: 12/931,932

(22) Filed: Feb. 15, 2011

(51) Int. Cl. A61M 3/02

(52) U.S. Cl. 604/279

(57) ABSTRACT

The Douche and Enema Syringe will hermetically attach to standard bottled water. The syringe is connected to the flexible tube and it's connected to the cap chamber. The rear pipe is also connected on the other side of cap chamber and it has an opening with a slightly similar dimension to the neck of standard bottled water that is available in the market. The rear pipe and flexible tube has an internal axial passage in fluid communication to the nozzle. The rear pipe has thin rings/threads around the pipe passage with diameters slightly larger than the neck opening of the water bottle it will be attached to, so that when the rings are pushed into the water bottle, it hermetically joints the two. It then provides a smooth flow of water spraying out from the nozzle holes use for douche or enema.

### BACKGROUND OF THE PRESENT INVENTION

Irrigation devices have gained wide use in cleansing and treating the vagina/anus. One such system previously known reusable douche/enema have been particularly inconvenient for the user when away from home due to the bulkiness of the equipment and the maintenance of cleaning the device due to the frequent lack of facilities necessary to perform these operations, especially when traveling.

Disposable douches and enema are also inconvenient to use due to its bulkiness and the need to assemble many parts during use.

In addition to the disadvantages of previous inventions, most disposable pre-mix douches/enema are not allowed on airplanes because of the evolving security threats to aviation. It is also too bulky to put inside the purse.

### SUMMARY OF THE INVENTION

This invention may be summarized at least in part with reference to its objective. It is the primary objective of the present invention to provide the most convenient, effective, sanitary, safe to carry, and handy way to douche/enema conveniently without the bulkiness of the kit. All you need to carry is the present invention! When you are ready to use it, just connect it to the any standard bottled water.

The present invention can be manufactured as a soft flexible material to allow the user to adjust the syringe to the most comfortable position to avoid discomfort or injury. Therefore, it doesn't limit the manufacturing material to plastic.

Also, this invention can simply be produced, providing inexpensive syringes for healthy vaginal/anus hygiene.

### DRAWING

### Figures

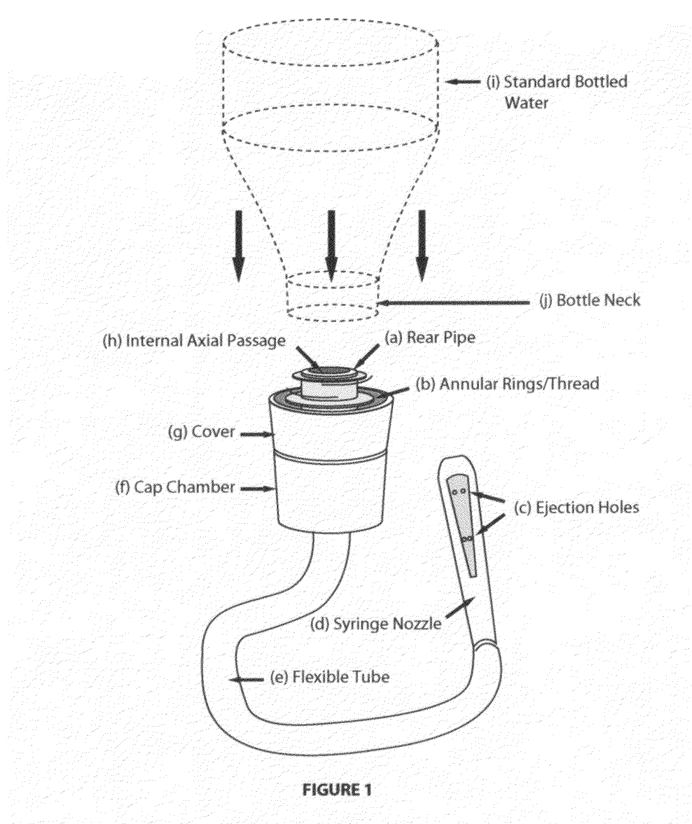

FIG. 1 Is the Perspective View of a syringe for vaginal irrigation in accordance with the preferred embodiment of the present invention. It shows combined syringe for fitting on bottled water, showing my new design. (The bottled water illustrated as broken lines is not part of the claimed design)

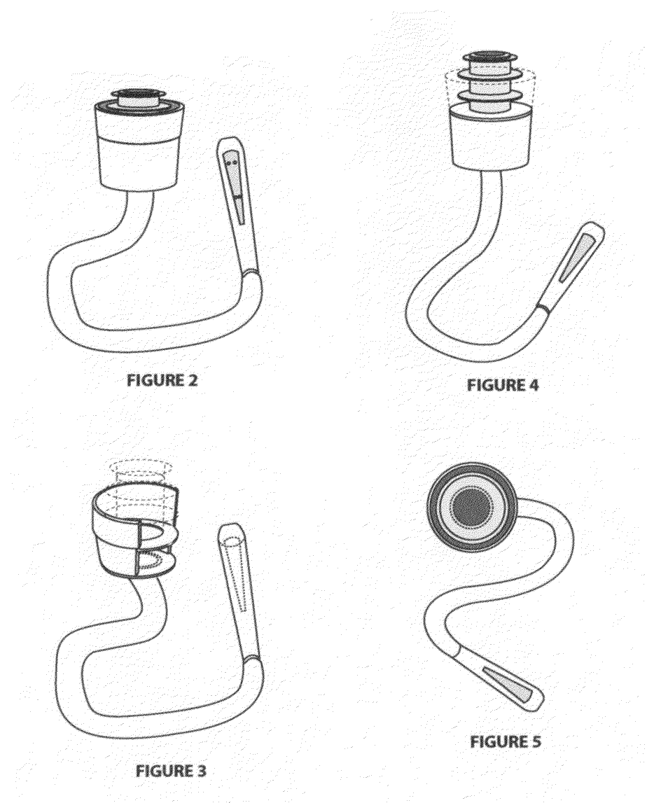

FIG. 2 The Side View of the syringe of FIG. 1

FIG. 3 The Exploded View of the syringe of FIG. 1

FIG. 4 The Perspective View without the cover of the syringe of FIG. 1

FIG. 5 The Top View of the syringe of FIG. 1

### DRAWINGS

### Reference

a. Rear Pipe
b. Annular Rings/Thread
c. Ejection Holes
d. Syringe/Nozzle
e. Flexible Tube
f. Cap Chamber
g. Cover
h. Internal Axial Passage
i. Bottled Water
j. Neck of Bottled Water

### DETAILED DESCRIPTION OF THE INVENTION

Referring to the drawings and particularly FIG. 1, the preferred embodiment of the invention, generally designated FIG. 1-A and B is illustrated that the Rear Pipe and Annular Rings/Threads is provided for sealing Present Invention to any bottled water. The cap chamber (f) has two portions, a lower portion where the rear pipe (a) is connected and upper portion where the flexible tube (e) is connected. The rear pipe (a) has plurality of thin, resilient annular rings/threads (b) affixed around it that seals the neck (j) of bottled water (i). The flexible tube (e) is where the syringe (d) connected. The rear pipe (a) and flexible tube (e) has internal axial passage (h) through which the fluid will pass from the bottled water (i) to the ejection holes (c). When the rear pipe (a) of the cap chamber (f) is forced into the neck (j) of bottled water (i) the rings/threads (b) are compressed against the inside surface of the neck (j) of bottled water (i), and sealing the internal axial passage (h). The syringe/nozzle (d) comprises a hollow vaginal or anus insert. When the present invention is completely connected to the neck (j) of the standard bottled water (i), the syringe/nozzle (d) is now ready to be inserted into the vagina or anus. When bottled water (i) is compressed by hand, the water due to pressure flows from standard bottled water (i), to the rear pipe (a), to the cap chamber (f), to the flexible tube (e), to the syringe (d) then flows through the ejection holes (c). Suitable pressure may then be applied to provide the desired flow rate of

water until the standard bottled water (i) is empty. The syringe (d) may be then be removed and disposed.

As described above, the present invention is only one piece, which has a Pipe that is inserted inside the neck of bottled water to seal it. The cap chamber has two portions, a lower portion where the rear pipe is connected and upper portion where the flexible tube is connected. The rear pipe has plurality of thin, resilient annular rings/threads affixed around it that seals bottled water. The flexible tube is where the syringe connected. The rear pipe and flexible tube has internal axial passage through which the fluid will pass from the bottled water to the ejection holes. When the rear pipe of the cap chamber is forced into the neck of bottled water the rings/thread are compressed against the inside surface of the neck of bottled water, and sealing the internal axial passage. The syringe comprises a hollow vaginal/anus insert. When the present invention is completely connected to the neck of the standard bottled water, the syringe is now ready to be inserted into the vagina or anus. When bottled water is compressed by hand, the water due to pressure flows from standard bottled water, to the rear pipe, to the cap chamber, to the flexible tube, to the syringe then flows through the ejection holes. Suitable pressure may then be applied to provide the desired flow rate of water until the standard bottled water is empty. The syringe may be then be removed and disposed.

While a full and complete description of the invention has been set forth in accordance with the dictates of the patent Statues, it is to be understood that the invention is not intended to be limited to be specific from herein shown.

Accordingly, modification of the invention may be shown without departing from the spirit hereof or the scope of the accompanying claims.

1. Douche and Enema for vaginal or anus irrigation, compromising:
a. The Present Invention is only one piece which has hermetic pipe that seals any bottled water
b. The Chamber Cap has two portions, a lower portion where the rear pipe is connected and upper portion where the flexible tube is connected
c. The Lower Portion of the Chamber Cap is the rear pipe and has internal axial passage through which the fluid will pass from the bottled water
d. The Upper Portion of the Chamber Cap is the flexible tube. The Syringe is connected to the flexible tube. The Flexible tube also has internal axial passage through which the fluid will pass from the bottled water to the syringe
e. The resilient annular ring/thread around the rear tube has a greater dimension than the inside diameter of the neck of Bottled water.
f. When the rear pipe is forced into the neck of the bottled water the rings/threads are compressed against the inside surface of the neck container, and sealing the junction between the Hermetic Cap and the Bottled Water
g. The FIG. 1 can be manufactured in Plastic (polyethylene); however, It is not limited to plastic and can be manufactured in rubber, flexible/plastic or in any other materials.
h. The ejection holes in FIG. 1 (c) can be manufactured with plurality of apertures.

2. The FIG. 8 shows that claim 1 can be manufactured solely as a connecting pipe without the outer cover (FIG. 1-g)

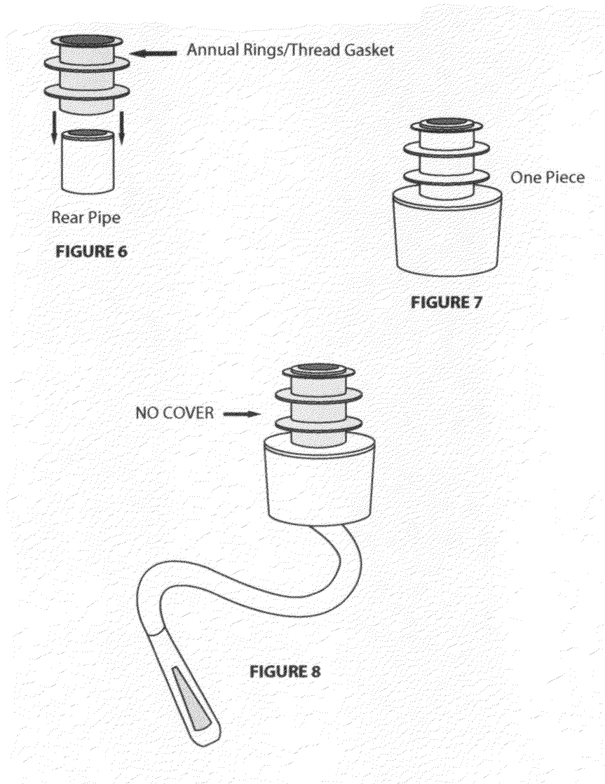

3. FIG. 6 shows that the claim 1 Annular Rings/thread Gasket and Rear Pipe can be manufactured as separate parts or FIG. 7 shows that the claim 1, Annular Rings/Thread and the Rear Pipe can be molded as one piece.
